(19) United States
(12) Patent Application Publication
(10) Pub. No.: US 2024/0335292 A1
(43) Pub. Date: Oct. 10, 2024

(54) MINIMALLY INVASIVE DIRECT POSTERIOR SACROILIAC JOINT FUSION

(71) Applicant: Frank H. Boehm, JR., New Hartford, NY (US)

(72) Inventor: Frank H. Boehm, JR., New Hartford, NY (US)

(21) Appl. No.: 18/623,462

(22) Filed: Apr. 1, 2024

Related U.S. Application Data
(63) Continuation-in-part of application No. 17/559,669, filed on Dec. 22, 2021, now Pat. No. 11,944,548.
Application No. 17/559,669 is a continuation of application No. 16/312,866, filed on Dec. 21, 2018, now abandoned.
(60) Provisional application No. 62/353,828, filed on Jun. 23, 2016.

(51) Int. Cl. A61F 2/30; A61B 17/16; A61F 2/46

(52) U.S. Cl. CPC A61F 2/30988; A61B 17/16; A61F 2002/30136; A61F 2002/30995; A61F 2/4603

(57) ABSTRACT

A device for fusing bone segments especially adapted for a new approach to fusing bone segments or separate bones, the device uses a novel closure mechanism to draw bone segments closely together to promote greater bone fusion strength.

### CROSS-REFERENCE TO RELATED APPLICATIONS

This application is a continuation-in-part of U.S. patent application Ser. No. 17/559,669 filed Dec. 22, 2021, which is a continuation of U.S. patent application Ser. No. 16/312,866 filed Dec. 21, 2018, which is a national stage application of International Application serial number PCT/US2017/039007 filed Jun. 23, 2017, which claims priority to U.S. Provisional Patent Application Ser. No. 62/353,828, filed Jun. 23, 2016, the disclosure of which is hereby incorporated entirely herein by reference.

### BACKGROUND OF THE INVENTION

The sacroiliac joints (SIJS) have been of great interest to physicians, anatomists, physiologists and others who study the musculoskeletal structures for many centuries. They are large, complex joints which complete the pelvic ring by serving as the interface between the sacrum and the major bones of the pelvis-the left and right iliac bones. On each side, the ilium is one of the three bones that create the acetabulum and since the ilium serves as the interface between the pelvic ring and the sacrum, it transmits vector forces coming from the lower extremity through the hip joint and on to the SIJ.

The sacrococcyx is the most caudal part of the spine and serves as the “base” thereof. It consists of 9 fused vertebrae which adds to the complex morphology of the SIJ; moreover, it is through this base of the spine that downward forces from the upper torso are transmitted through the sacrococcyx to the SIJs bilaterally and thence to the pelvic girdle. Hence, the SIJs are functionally “portals” through which these very heavy [midline] downward forces are presented to and disseminate/equilibrate with the heavy upward forces from the lower extremities. Arguably, the SIJs are the most heavily loaded joints in the body, particularly as they are subjected to vector loads from both directions.

Again, from an anatomic perspective, this is a complicated joint that structurally and functionally is composed of two different classic joint morphologies. The joint's anterior portion is a true diarthrosis or gliding synovial plane joint; this has been confirmed by multiple different studies. The posterior aspect of the joint, on the other hand, is a syndesmosis composed of a heavy fibrocartilaginous joint.

Multiple pathologies are responsible for SIJ dysfunction including trauma, hypermobility, degenerative and/or osteoarthritic processes, and inflammation.

NIH reports have demonstrated the prevalence of SIJ dysfunction amongst patients with back pain, especially those who have undergone surgical fusion of the spine (more than 50% have SIJ dysfunction). It is thought that this may represent, at some level, a variant of the so-called “adjacent disc disease” which could perhaps more generally be referred to as “adjacent joint disease”. Numerous systems for fusing this joint have been developed and brought to market.

In comparing SIJ dysfunction to other joints, additional conundrums present themselves. When examining pathologic joints such as knees, hips, or shoulders, it has been established that loss of joint space, erosion of cartilage lining the joint, and “bone on bone” friction are the main causes of symptoms in these very mobile joints which are subject to almost constant ongoing movement; none of these mechanisms appear operative when attempting to understand the causes of pain arising the synovial SIJ in which for much of the joint, by definition, is “bone on bone.”

Surgical fusion of the SIJ often improves, and in many cases resolves discomfort arising from the SIJ. This would generally lead one to the inescapable conclusion that with degeneration, some form of instability of the joint likely contributes to symptoms. A slight variation on that theme is that at least when spinal fusion has been performed, it causes, euphemistically speaking, a biomechanical “overload,” of the SI joint which is somehow better managed by fusing the joint.

Therefore, what is needed is a device which clearly establishes the maximum amount of fusion mass bridging the component osseous elements. Such a device would be unique, useful, novel and nonobvious.

### BRIEF DESCRIPTION OF THE INVENTION

The invention relates to the general field of spinal surgery and specifically to a unique, useful, novel, and non-obvious method for achieving minimally invasive (MIS) direct posterior fusion of a target sacroiliac joint (SIJ). The target joint is identified, and after stripping the cartilaginous lining and decortication, an implant is secured between the ilium and sacrum. Upon deployment of the instrument, a central wing-like component rotates such that it is orthogonal to the long axis of the implant, the deployed component helps to lock the implant in place, reducing the chances of being “spit out,” posteriorly. Furthermore, the deployment of the wing-like central component creates additional bone graft promoting fusion.

The invention relates to the SIFs described previously but adds two further unobvious improvements wherein the blade is contained in a cage, and secondly improved blades having two blade assemblies around a central axis.

### BRIEF DESCRIPTION OF THE DRAWINGS

The drawings illustrate the features of certain embodiments of the invention. In the drawings, like numerals are used to indicate like parts throughout the various views. Thus, for further understanding of the invention, reference can be made to the following detailed description read in connection with the drawings, in which:

FIG. 1 is a posterior view of the pelvic girdle.

FIG. 2. shows a lateral view of the pelvic girdle, in particular illustrating the SIJ.

FIG. 3. Reveals an anterior view of the pelvic girdle.

FIG. 4 examines a transaxial view of the cranial aspect of the pelvic girdle. 8

FIG. 5A portrays an elevational view of the leading end of the implant in its non-deployed embodiment.

FIG. 5B is an elevational view of the top of the SIFS.

FIG. 6 is a patient positioned favorably to undergo implantation of a right sided SIFS.

FIG. 7 illustrates the first step in the implantation process with placement of a guide pin into the target joint space.

FIG. 8 demonstrates disposing the initial guide cannula over the pin that was placed in the joint.

FIG. 9 reveals a top oblique view of the initial guide cannula as it would relate to the target joint.

FIG. 10 is a lateral elevational view of the isolated working cannula.

FIG. 11 shows a lateral view of the working cannula being passed over the initial guide cannula.

FIG. 12 portrays a top view of the working cannula in position after removal of the initial guide pin and guide cannula, and prior to implantation of the stabilizing screws.

FIGS. 13A/B—“A” demonstrates an axial view showing the cannula after placement of the stabilizing screws; “B” displays a top view of the cannula with the stabilizing screws in place. 9

FIG. 14 displays an elevational view of the isolated drill guide.

FIGS. 15A/B—“A” is a top view of the drill guide in place within the working cannula; “B” is a lateral view of the drill guide in place with a drill having been passed through the cranial channel.

FIG. 16 demonstrates the box chisel in an elevational view.

FIGS. 17A/B—“A” shows a transaxial view of the pelvis with the box chisel in place, with the leading end coring out the remainder of the material in the joint space; “B” is a lateral view of this process.

FIGS. 18A/B/C illustrate an embodiment of the implant; FIG. 18A is an enlargement of the SIFS reversibly coupled to the leading end of the insertion device; FIG. 18B displays the configuration of the deployed implant; FIG. 18C shows an elevational view of the entire insertion device with a non-deployed implant coupled to the leading end.

FIGS. 19A/B/C are lateral views of the embodiment of the SIFS being passed into position and deployed.

FIG. 20 illustrates a system for preparing the joint space for fusion.

FIG. 21 displays a template used to identify the surgical landmarks preoperatively.

FIG. 22 is an embodiment of the SIFS with a cage carrier and a dual block blade with two side blades extending from a central axis engager and a connecting blade between the two side blades.

FIG. 23 demonstrates another alternative embodiment of the SIFS.

FIG. 24 illustrates yet another alternative embodiment of SIFS.

FIGS. 25A/B displays embodiments of the SIFS using no cage.

FIGS. 26A/B are additional embodiments of the a SIF.

FIG. 27A reveals a frameless SIFS in final position.

FIG. 27B shows two SIFS units stabilizing a target SIJ.

FIG. 28 examines an alternative method of implanting an SIFS.

FIGS. 29A/B portray an illustration of the use of the SIFS in a long bone fracture suffering from non-union.

### SUMMARY OF THE INVENTION

This written description uses examples to disclose the invention, including the best mode, and also to enable any person skilled in the art to practice the invention, including making and using any devices or systems and performing any incorporated methods. The patentable scope of the invention will be defined by the claims.

The invention is to provide a unique, useful, novel, and nonobvious device and method for achieving a fusion of the sacroiliac joint utilizing a minimally invasive direct posterior approach. This fusion is achieved by the implantation of a stabilizing device which will at once stabilize the joint as well as promote fusion. This implant is hereinafter referred to as the Sacro-Iliac Fusion Stabilizer (SIFS). It is anticipated that this implant would be fabricated from surgical grade Titanium, in the preferred embodiment, but can also be alternatively composed of surgical grade Stainless Steel, as well as any alloy, such as Nickel, Cobalt, Chromium, Molybdenum, or any other metal or metal alloy known or acceptable to the art. Additionally, an embodiment composed of cadaveric bone, porcelain, PEEK in all its iterations, or any other nonosseous substance acceptable to this use within the general field of spinal surgery is of course within the spirit and scope of the invention.

Implantation is performed using a direct minimally invasive procedure from a posterior approach. The initial step in this implantation is positioning the patient on the operating table in the position best suited for such a procedure. Ideally, this is a semi-prone position with the proposed surgical side down and supporting the patient's torso with pillows/supporting cushions. In the preferred positioning, the patient can be rotated so that the target joint is orthogonal to the floor. This reduces the angles that the instruments are needed to be brought into in order to perform such a procedure which may increase the comfort level of some surgeons. In other instances, the procedure can be performed with the patient is a direct prone position with the surgeon willing to operate at somewhat of a bias to the patient. This would ultimately refer to the surgeon's preference. Regardless of these considerations, a more important concern would be to have intraoperative imaging, whether it is intraoperative fluoroscopy, transaxial imaging (CT or MRI), or computer assisted intraoperative imaging. This is a critical component of the procedure.

The target SIJ is placed using intraoperative imaging, with the surgeon “free-handing,” the direction of the guide pin into the joint. In the preferred embodiment, the leading end of the guide pin is composed of stainless surgical steel, or any other metal or other radiopaque substance known or acceptable to the art. The remainder of the guide pin is composed of hard plastic or any other radiolucent substance. This would make identifying the leading end much easier on intraoperative imaging.

A template that can be used intraoperatively to guide the user in the initial placement of the guide pin. This template consists of a sterile plastic sheet with radiopaque 14 markings that emulate the SIJ. The template is also provided with guide apertures which direct the guide pin. By aligning the template with the fluoroscopic image, the guide pin can be disposed through the most appropriate guide aperture, and in that way readily passed into the target joint.

In some or all of the embodiments disclosed herein, following the placement of the guide pin, an initial guide cannula is disposed over the guide pin.

In some or all of the embodiments disclosed herein, the working channels of the working cannula are configured such that they are provided with central internal channels which are configured such that the surfaces of these channels are slightly larger than and precisely recapitulate the external surfaces of the guide cannula such that the working cannula is precisely disposed over the guide cannula.

In some or all of the embodiments disclosed herein, the working cannula is provided with a leading end which is, in turn, provided with a shorter side and a longer side; the shorter side is configured to be brought against the prominent iliac crest, thus circumventing a problem that often confronts surgeons attempting to approach SIJ fusion from a posterior approach. The longer side is designed to be brought against the corresponding site on the sacrum.

In some or all of the embodiments disclosed herein, the leading end of the working cannula is provided with anchoring screws which are configured to be inserted into the corresponding osseous 16 structures such that one of these screws are anchored into the sacrum while the other is anchored into the ilium.

In some of the embodiments disclosed herein, the working cannula is provided with a pivotable base according the cannula the ability to be repositioned such that the working channels are redirected cranially or caudally, ultimately accommodating the implantation of multiple SIFS implants.

In some of the embodiments disclosed herein, the SIFS implant includes a rotating component which has been provided with deployable extensions added to the blades, whereupon with full 19 deployment, there is a greater extension of the wings into the osseous substance of the ilium and sacrum.

In some of the embodiments disclosed herein, the rotating component is not monolithic; rather, the two blades are independent units, and as such, extend the entire length of the implant, rather than half of the length, as is the case with a monolithic blade. After the initial positioning of the SIFS within the joint, the blades are slidably repositioned such that they are coupled to the central axis, at which point they are rotated, achieving maximum depth into the respective bones.

In some of the embodiments disclosed herein, the blades are again independent, rather than a monolithic structure, with the blades serving as completely independent units which are simultaneously deployed, again ultimately achieving a deeper penetration into the target bony structures.

In some or all of the embodiments disclosed herein, a monolithic blade serves as the SIFS, without any encasing component. 20

In some or all of the embodiments disclosed herein, a plurality of rotating blades which are coupled by a central axis, and which are not contained within an encasement are utilized as the SIFS.

In some or all of the embodiments disclosed herein, a single, long blade is positioned through a minimally invasive incision, and rotated into position.

In some or all of the embodiments disclosed herein, the blades referred to above may include embodiments such as that described in the preferred embodiment, in which large apertures are present within the major face of the blade. Additionally, there may be blades in which multiple small apertures are present, as well as complex blades in which multiple apertures are present which are continuous with chambers within the central portion of the blades.

In some or all of the embodiments disclosed herein, it can be anticipated that other uses of any or all of the embodiments can be conceived, including using said embodiments for primary treatment of fractures at any other site, as well as treatment of fractures in which nonunion has occurred.

### DETAILED DESCRIPTION OF THE BACKGROUND DRAWINGS

The invention will be best understood if the reader is provided with a fundamental understanding of the pertinent anatomy, and the relationships of various landmarks of the osseous to key soft tissue structures of the spine. These images are representations of the lumbar spine, as the many objects of this invention is to a unique, useful, novel and nonobvious approach to the target vertebra, wherein a lateral interbody discectomy and fusion is achieved. This, however, recognizes that not all the critical neural and soft tissue structures have been included in these drawings and that despite their exclusion, these structures must be accounted for within the process of implantation of the invention. Although these soft tissue structures (with the exception of the intervertebral discs and exiting nerve roots) are not illustrated herein, the images will, nevertheless, sufficiently demonstrate the relationships of critical soft tissues such as the spinal cord and nerves to the bony anatomy. When relevant, these structures will be referred to by name in these initial images.

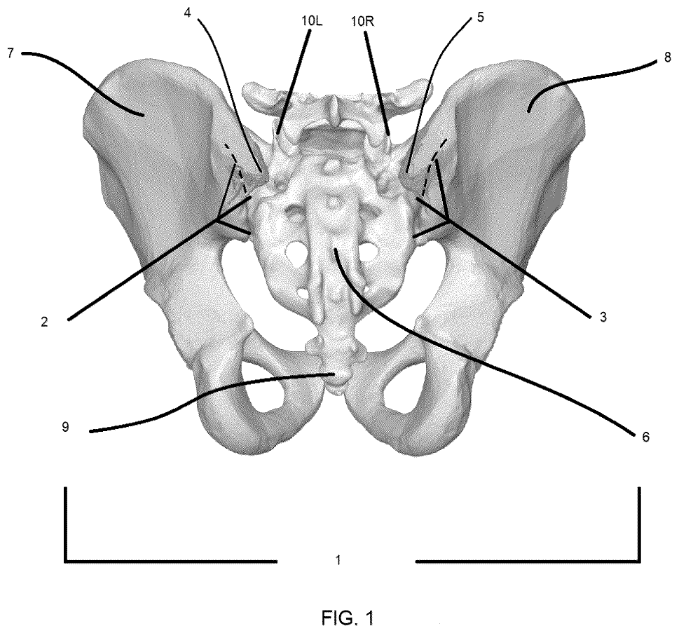

FIG. 1 shows a posterior view of the pelvis, also known as the pelvic girdle 1, including the sites of the Left 2 and Right 3 SIJS, which are partially obscured by the left 4 and right 5 posterior iliac crests, the obscured joints indicated by the curved interrupted lines. The sacrum 6 is the posterior central portion of the pelvic girdle 1. The large expansions of the left 7 and right 8 anterior iliac crests are also seen from this perspective. The coccyx 9 can be seen at the bottom of the sacrum. The top of the sacrum is referred to in standard parlance as S1 (although the sacrum is technically fused into 1 bone, this is the vestige of the fetal S1 which was separate); the articulations of S1 with the L5 are also illustrated herein, 10L, R.

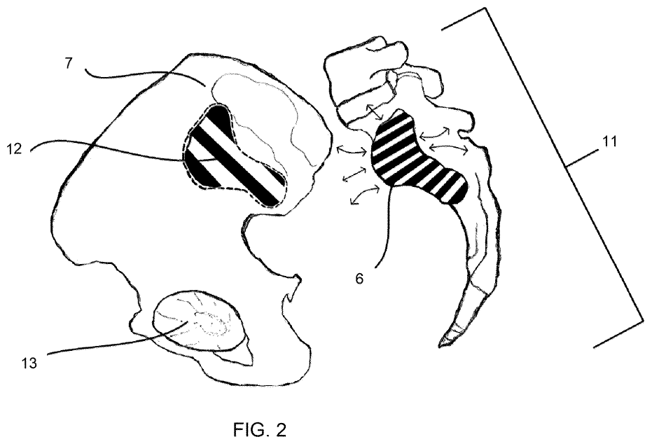

FIG. 2 is a left lateral view of the pelvis 1, showing the disarticulated sacrococcygeal segment 11 and, most importantly, demonstrating the configuration of the articulation of the left side of the sacrum 6 and the left ilium 7, this articulation comprising the left sacroiliac joint (SIJ), not seen in its articulated form in this view. The SIJ is classically referred to as an “auricular-shaped joint,” and this configuration is readily appreciated in the disarticulated format herein. In this illustration, the reader is viewing ilium from the lateral side, as demonstrated by the presence of the acetabular cup 13. The iliac articulation 12 is on the medial 23 side of the pelvis, as indicated by the encasing interrupted line; the iliac articulation is portrayed by the vertically oriented hatched area 12. The sacral side 6 is demonstrated by the horizontal cross-hatched area; it is anticipated that the joint space is approximately 1-2 mm in width, with the amount of motion depending upon a number of factors, including age, sex, genetic factors, as well as in association with certain disease states. The general planes of the gliding-type motion of the joint are portrayed by the arrows; the most common movements of the joint are complex gliding movements, as indicated by the curved arrows, combining movements along the anterior-posterior plane as well as along the craniocaudal plane. “Pure,” A-P or craniocaudal movements are possible, but thought to occur less often. Movements of the sacrum and ilium towards/away from each other probably also occur, as suggested by the curved arrows below the illustration, but such movements would not be readily appreciated in this perspective.

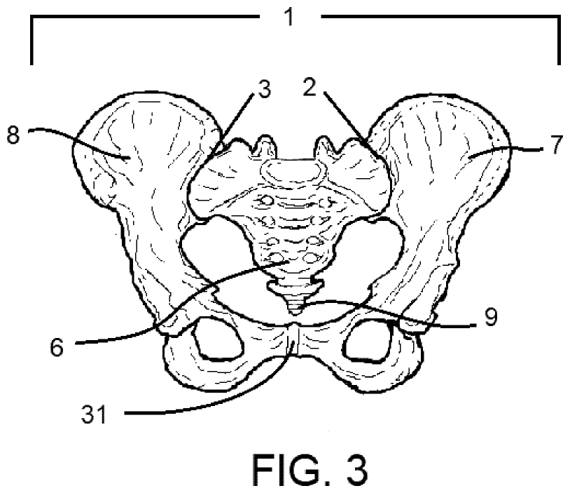

An anterior view of the pelvic girdle 1 is seen in FIG. 3, again demonstrating the right 3 and left 2 sacroiliac joints along their anterior aspects. The sacrum 6 is prominent in this view, as it is recalled that the top part of the sacrum is referred to as the sacral promontory. The intra- and retroperitoneal visci and structures are not seen, but include the 24 large vessels (iliac vessels, aorta and vena cava) are directly in front of the upper sacrum, as well as small intestines and colon, the components of the urological system, and the lumbosacral plexi. The presence of these structures underscores the importance of not going too deep with such an injection. Although it seems impossible to pass through this [largely narrow] joint, reports of orienting the needle too cranially resulting in “skiving off,” the joint and into such structures exist. Lateral to the SIJS are the very prominent iliac crests 7, 8. Also appreciated are the coccyx 9, and the symphysis pubis 14. The fibrous symphysis pubis represents that anteriormost limit of the pelvic ring; movements of the symphysis pubis may reflect reciprocal movements of the joint.

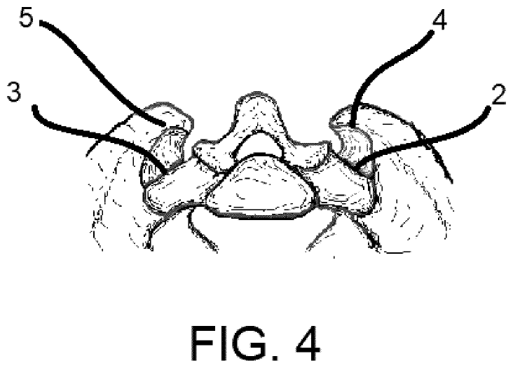

To further the reader's understanding of the pertinent anatomy, a cranial transaxial perspective of the sacrum and its bilateral articulations with the Ilia is provided in FIG. 4. The posterior aspects of this anatomy appear Here, it can be seen that the left 2 and right 3 SI joints are directed laterally in an oblique fashion in their course from their posterior aspect to the anterior most part of the joint. The overhang of the Posterior 25 Iliac Crests 4, 5 is best demonstrated in this view, revealing the challenge in reaching the joint from a posterior approach.

### DETAILED DESCRIPTION OF THE DRAWINGS DISCLOSING THE INVENTION

The invention is best understood by studying the following detailed descriptions in conjunction with the context of the accompanying images, wherein like reference numbers refer to like structures, in accordance with common practice. Also in accordance with common practice, the structures illustrated are not necessarily drawn to scale, nor can inferences of scale be developed with respect to such drawings. The embodiments presented and illustrations herein are general representations of the invention and are not, nor can they be, construed to be restrictive.

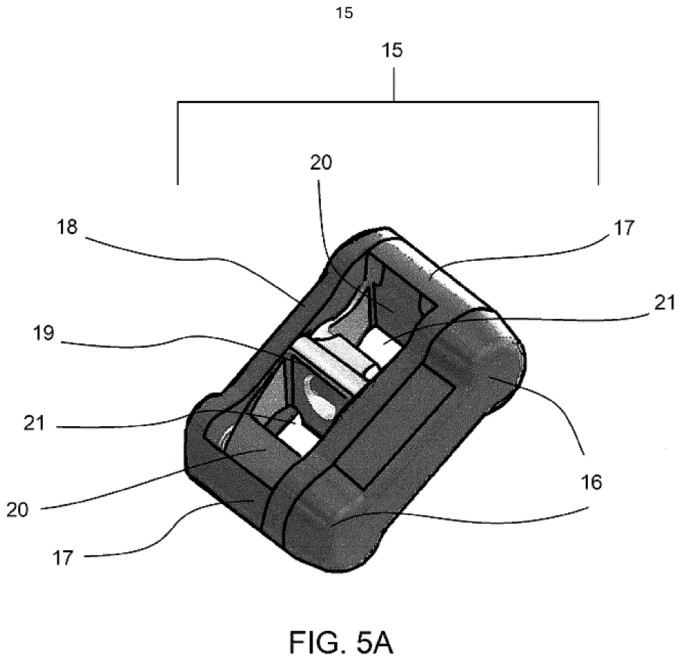

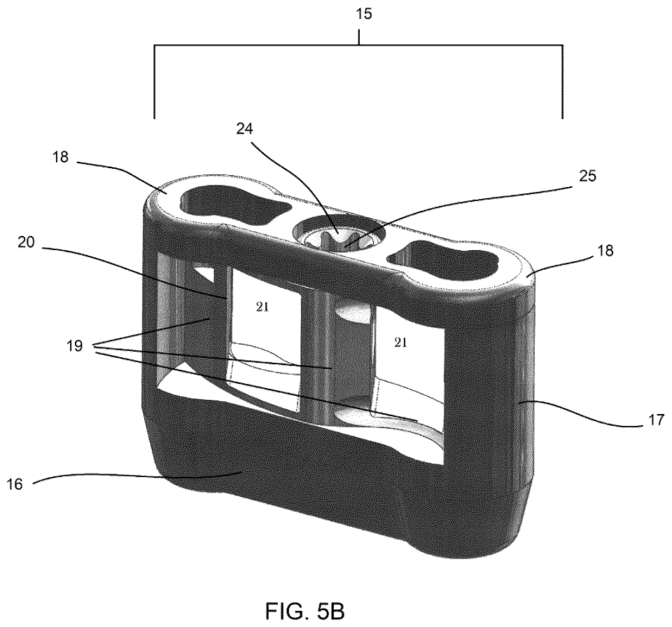

An elevational view of the leading end 16 of the SIFS 15 is seen in FIG. 5A. As can be appreciated, implant/SIFS 15 is provided with a leading end 16, a central deployable segment 17, and a trailing end 18. Furthermore, the leading end 16 is somewhat tapered so that it can be more easily insinuated into the target joint space, and may be provided with a plasma-sprayed coating, or may be provided with any other enhancement 26 to improve fusion. This, in fact, may apply to the outer coating of any other segment of the SIFS 15. It is also noted that the central segment is provided with a rotating component 19, which is critical to the functions of the SIFS. This rotating component 19 rotates around a central axis (not seen in this view), with each end further provided with blades 20 designed to cut into the cancellous bones of the ilium and sacrum. Apertures 21 positioned within these blades 20 direct the cancellous bone harvested by rotation of the blades 20 into the central portion of the SIFS, as well as into the central area between the sacrum and ilium; as already disclosed, the implantation of the SIFS includes removing the cartilage and decorticating the arthrodial surfaces of the Ilium and sacrum.

The same implant 15 is seen in an elevational view of its trailing end 18 in FIG. 5B. The trailing end 24 of the central axis 23 which actuates the rotating component 19 of the implant 15 can be seen. It is noted that this trailing end 24 is provided with an insertion area 25 where a rotating leading end of the insertion instrument, which is not shown here, can insert in order to actuate the mechanism. Upon actuation, the blades 20 27 of the rotating component 19 will be brought into a position in which they are approximately orthogonal to the implant; when this happens once the implant 15 has been positioned within the joint, the blades 20 are driven into the cancellous bone of the sacrum and ilium. Apertures 21 direct bone harvested from these osseous structures by the action of the rotating component 20 into the central portion of the implant 15. The rotating component 19 is positioned within the central segment 17 of the Implant 15. The leading end 16 can be appreciated in this view as positioned to be directed into the SIJ. The SIFS has a cage carrying the central rotating axis, a body with a first and second opening allowing rotation of the blades about the axis; and a first and second blade each with two extending blades and a top rotating blade joining the two extending blades as shown in the FIG. 5B.

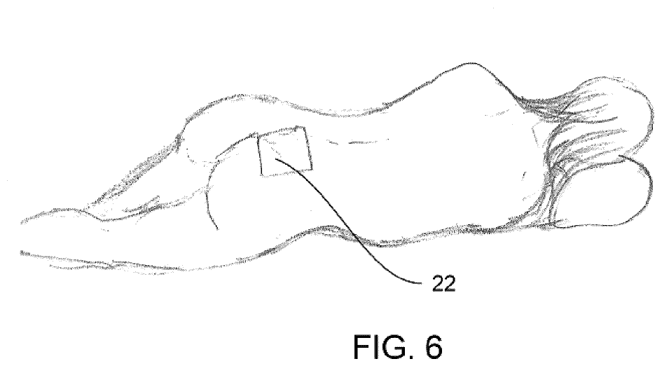

Pictured in FIG. 6 is a patient about to undergo a right sided SIJ fusion, positioned in the semi-prone position. This is an acceptable position for this procedure. The greatest advantage is that if the patient is positioned with the assistance of fluoroscopy, a position can be achieved in which the joint is essentially orthogonal to the floor, making the ergonomics of handling the various operative instruments very easy, as they will be held in a full vertical position. Many surgeons seek to achieve such ergonomics for surgical procedures, particularly ones which require 28 the application of any force against bone. The disadvantage of this procedure is primarily that it can be anticipated that the patient will be managed with either general anesthesia or heavy sedation; in either instance, if great care is not taken to secure the patient to the OR table, the patient could theoretically slide off the table during the procedure, a dissatisfying experience for all. In this image, the proposed operative field is indicated by the square 22.

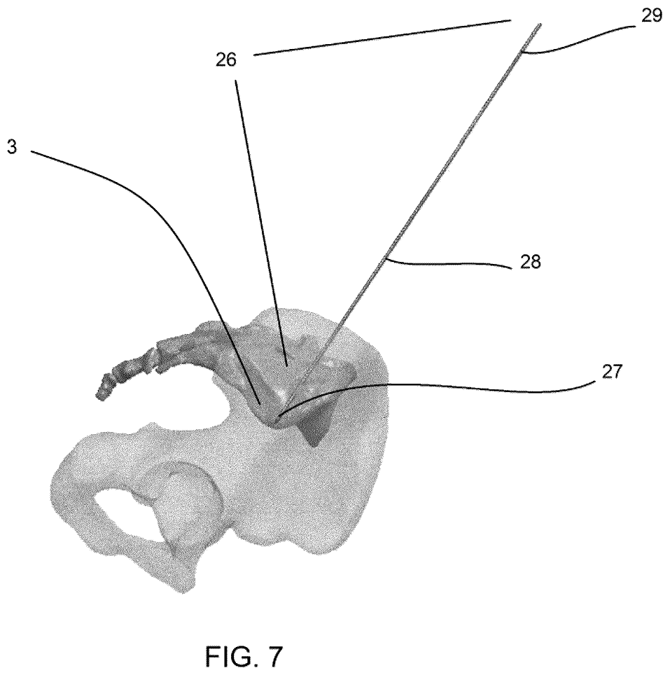

The first step of the procedure is to insert a guide pin 26 into the target joint. In FIG. 7, the ilium has been removed to show the sharpened leading end 27 of the guide pin 26 in position against the sacral aspect of the right SIJ 3. The shaft 28 of the guide pin is of sufficient length so the trailing end 29 can accommodate the guide cannula, to be inserted in the next step. The trailing end 29 is of course that part of the guide pin 26 which is actuated by the surgeon. The placement of the pin is verified by fluoroscopy.

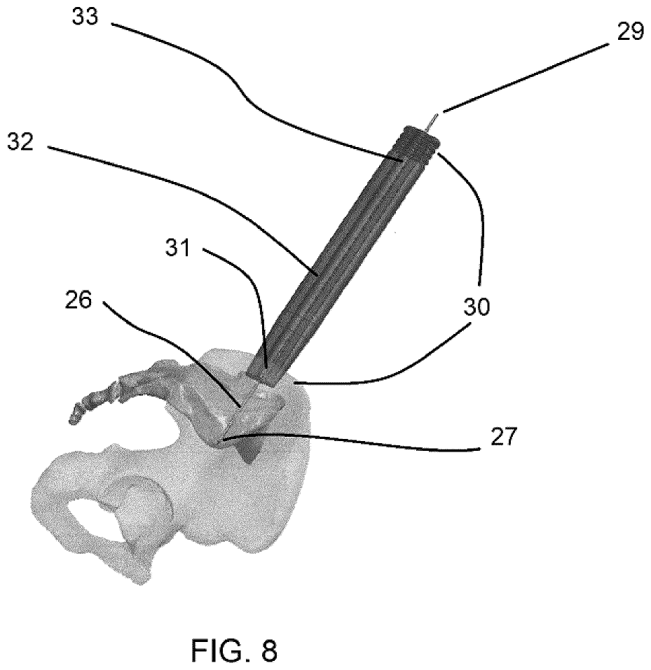

Upon verification of good positioning, a guide cannula 30 is passed over the guide pin 26, the trailing end 29 of which is seen protruding past the trailing end 33 of the guide cannula 30 in FIG. 8. In 29 positioning the cannula 30 into the SIJ, the leading end 31 is implemented as an obturator to enter into the posterior capsule of the SIJ. Again, the shaft 32 allows for sufficient length so that the surgeon can comfortably position and manipulate the trailing end 33 after the entire device has been positioned. In this view, the leading end 27 of the guide pin 26 is maintained within the joint space at this point in the procedure.

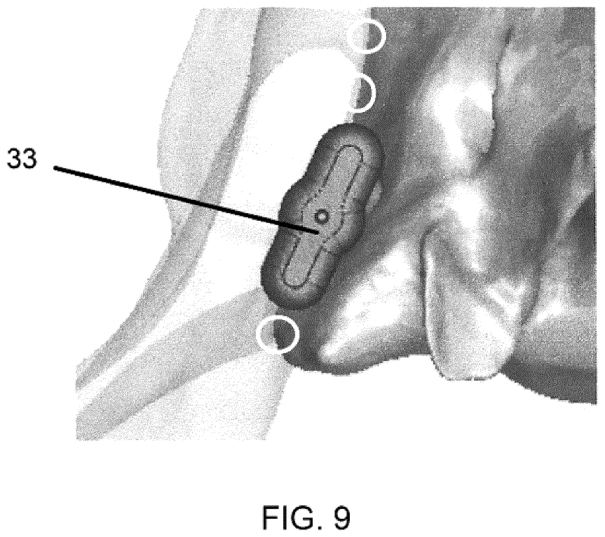

An important aspect of the guide cannula is that it serves as a template for the passage of the working cannula. This may be best appreciated in FIG. 9, which is a top view of the guide cannula in place. It can be noted that the sacrum is darker and drawn in relief with the joint proper being demonstrated by the white circles. The trailing end 33 of the guide cannula is seen in the top view, where it is noted that the cannula is more robust in the middle, with elongated cranial and caudal ends; these are slightly different in size, which would in turn specify the orientation of the working cannula.

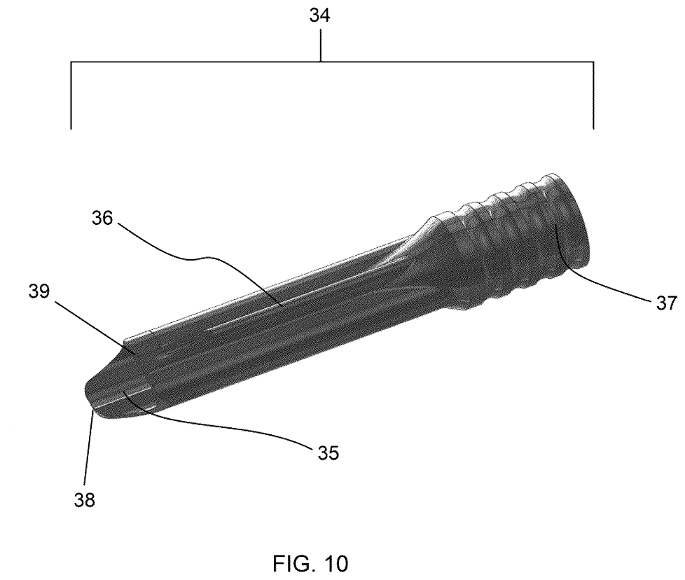

FIG. 10 shows a lateral view of the isolated working cannula 34, wherein key features of the cannula are seen; principally, the leading end 35 is noted to have a long/sacral limb 38, which is designed to be seated against the sacrum, while the short/iliac limb 39 is specifically 30 configured to be brought against the prominence of the posterior iliac crest. This configuration creates a unique, useful, novel and nonobvious design which assures that the channels of the working cannula are directed towards the target SIJ.

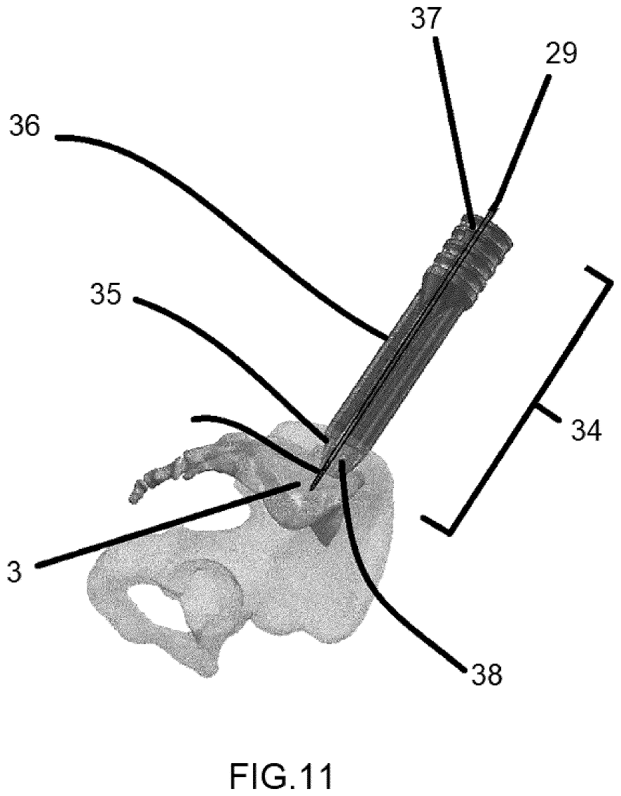

The uniqueness of the working cannula 34 can be further appreciated in FIG. 11, which portrays a lateral view of the cannula 34 functionally positioned. As before, the ilium has been subtracted to better illustrate the cannula 34. On notes that the cannula 34 has been disposed over the guide pin 26, which is still in the target [right] SIJ 3. As disclosed in the previous paragraph, the leading end 35 is provided with a sacral leg 38 which is brought against the sacrum (which is a deeper structure in comparison to the ilium). The short leg, also referred to as the iliac leg, is not well seen in this perspective, as it is cast against the rest of the cannula 34. The shaft 36 again provides adequate length to the cannula 34. The trailing end 29 of the guide pin 26 is seen protruding from the trailing end 37 of the working cannula 34. The pin and guide cannula will be removed after satisfactory positioning of the cannula 3.

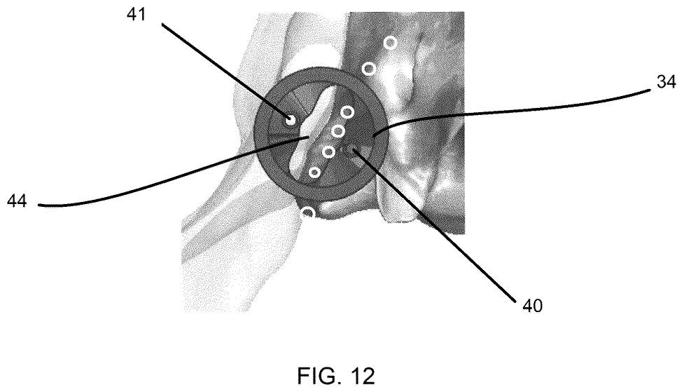

Upon positioning the working cannula, the guide pin and guide cannula are removed. It would be the surgeon's choice regarding 31 removing these prior to placing the stabilizing screws, but for illustrative purposes, in this image the cannula 34 is seen in a top view in FIG. 12, the pin and guide cannula have been removed revealing the working channel 44 centered over the target SIJ (indicated by the line of white circles). One takes note that the configuration of the working channel recapitulates the exterior configuration of the guide cannula. The stabilizing screws have not yet been placed, but the sacral 40 and iliac 41 screw tracts are clearly seen.

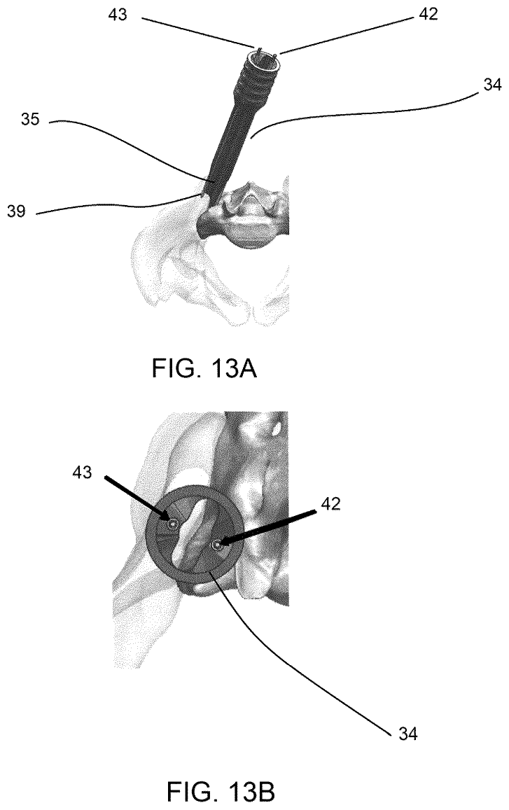

FIG. 13A further illustrates the value of the design of the cannula 34, with the leading end 35 provided with the sacral and iliac legs. The sacral leg 39 can be seen stabilizing the cannula 34 against the prominence of the iliac crest and assuring that the cannula is maintained in its position centered over the target SIJ. The sacral leg is not well appreciated in this view. What can be seen is how maintaining the cannula in this position will direct the implant along the course of the joint.

The top view in FIG. 13B shows the stabilizing screws 42, 43 now having been positioned and maintaining the cannula 34. At this point, the physician would obtain another radiologic image to be certain that the cannula is centered over the joint, prior to introducing the drill guide 32.

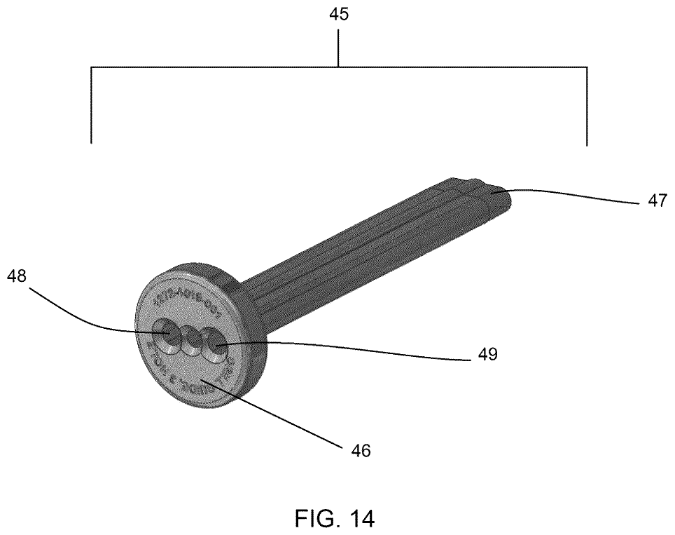

The drill guide 45 is configured to be reversibly coupled with the trailing end of the working cannula and is examined in the elevational perspective in FIG. 14. Provided herein is a monolithic unit with a leading end 47, which is inserted into the working channel of the working cannula, and a trailing end 46 through which the surgeon will dispose through the drills to complete the procedure. The cranial channel 48 and the caudal channel 49 will stabilize the drill bits.

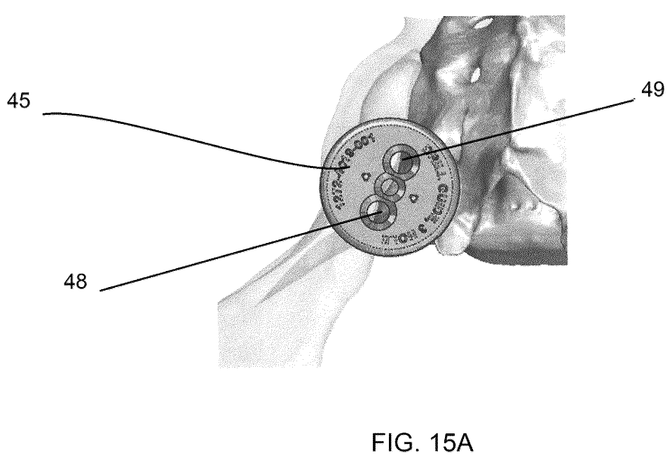

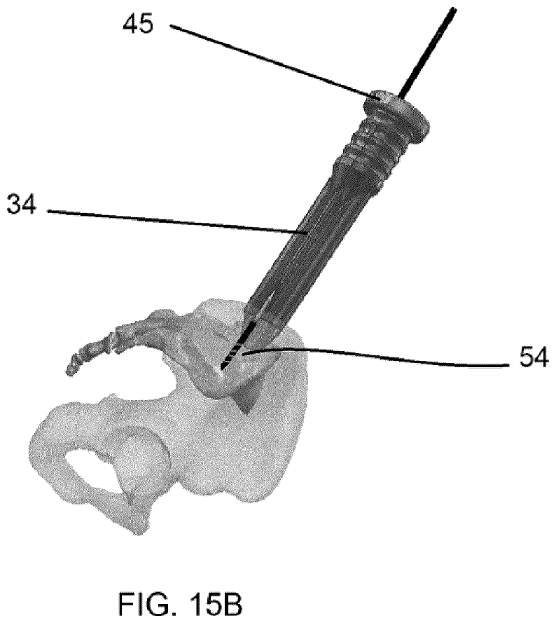

In FIG. 15A, it can be seen how the drill guide 45 is fitted into the trailing end 37 of the working cannula 34. A separate drill guide is necessary because the guide cannula must first be removed so that the drill guide 45 has access to the working channel. In the preferred embodiment, the cranial 48 and caudal 49 channels are distinct and the contours of the working channel and the drill guide permit only one manner by which the drill guide 45 can be inserted into the working cannula.

FIG. 15B gives the reader a lateral perspective of the drill guide 45 in place within the working cannula 34, with a drill bit 54 passing through the working channel and into the target joint. The ilium has again 33 been subtracted from this view. The drills can be either hand or power driven.

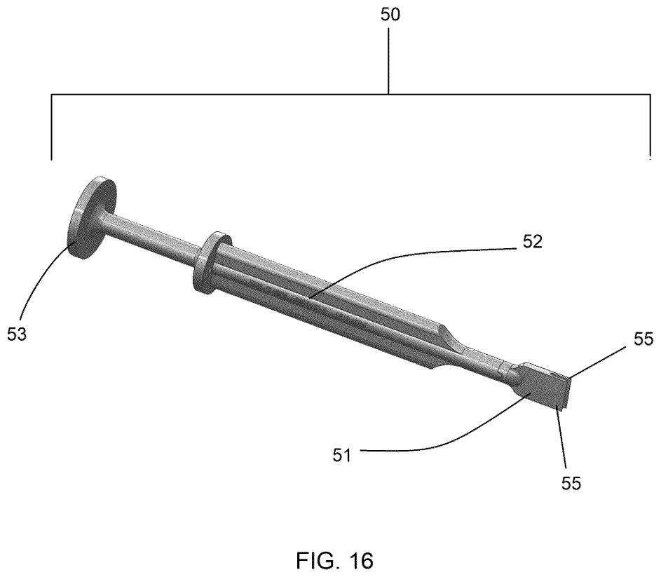

After drilling out the joint, which presumably denudes the cartilaginous surfaces as well as decorticating the participating bony surfaces, the drill guide is removed and a box chisel is introduced. An elevational view of the box chisel 50 is seen in FIG. 16, where it is noted that the instrument is a monolithic device provided with a leading end 51, which is provided with two chisel blades 55 which create a space between them. There is also a shaft 52 continuous with both the leading end 51 and the trailing end 53; the device is actuated by the trailing end 53.

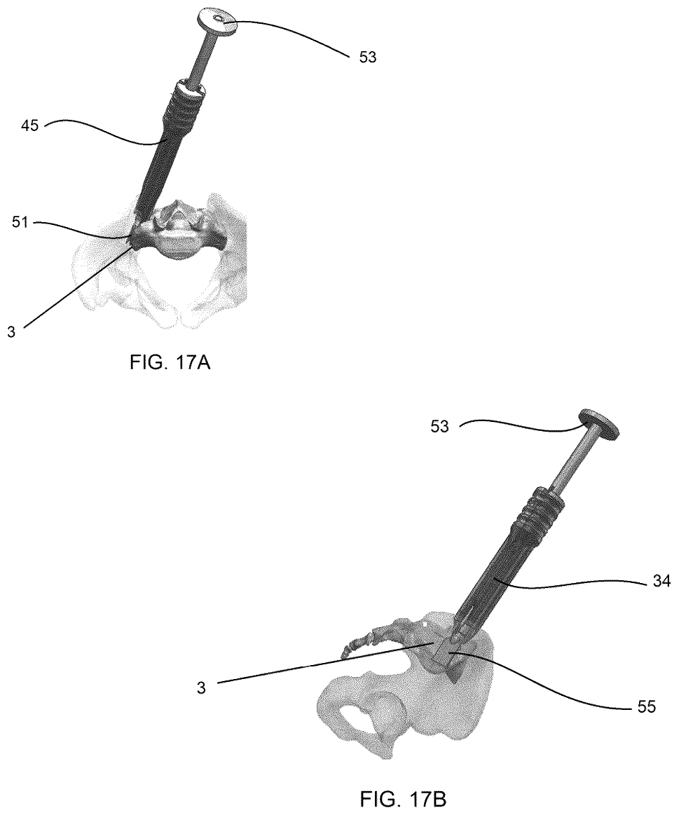

The leading end 51 of the box chisel 50 is disposed through the working cannula 34, with an objective to clear further debris from the joint space prior to implantation of the SIFS. This is well appreciated in FIG. 17A, a cranial perspective of a transaxial view of the pelvis in which the box chisel has been introduced into the right SIJ 3. The surgeon controls the chisel by actuating the trailing end 53.

FIG. 17B is a lateral view of the box chisel 50 within the joint 3; the ilium has again been subtracted in this view. The blades 55 are much better seen in this perspective, and their ability to reduce debris in 34 the joint can be more fully seen. The working cannula 34 and the trailing end 53 of the box chisel 50 are also portrayed in the picture.

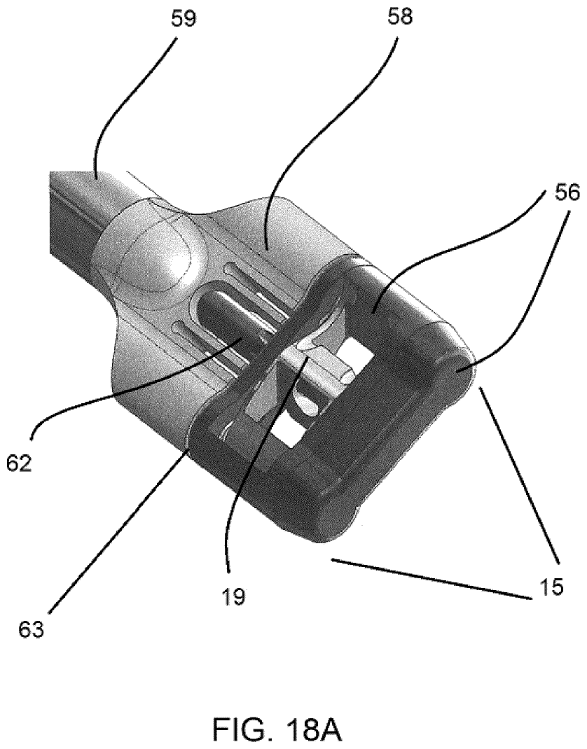

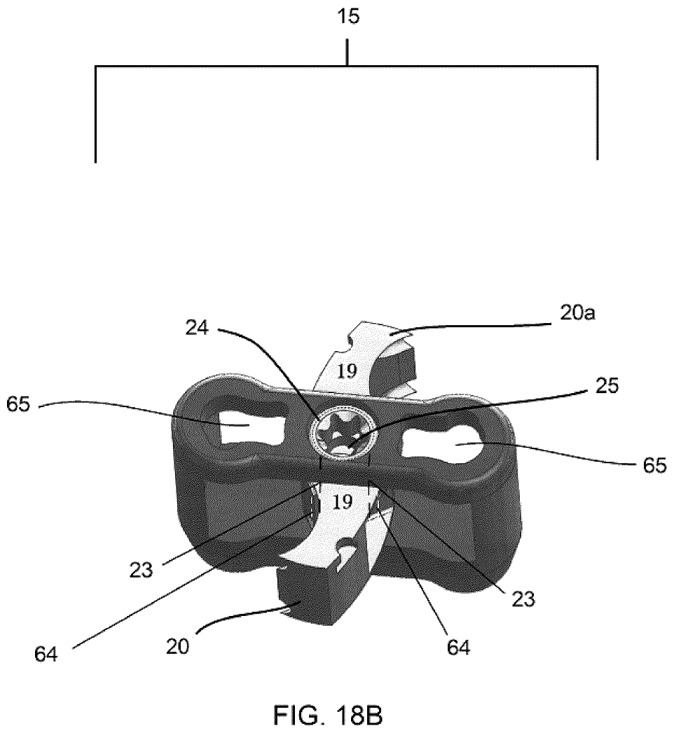

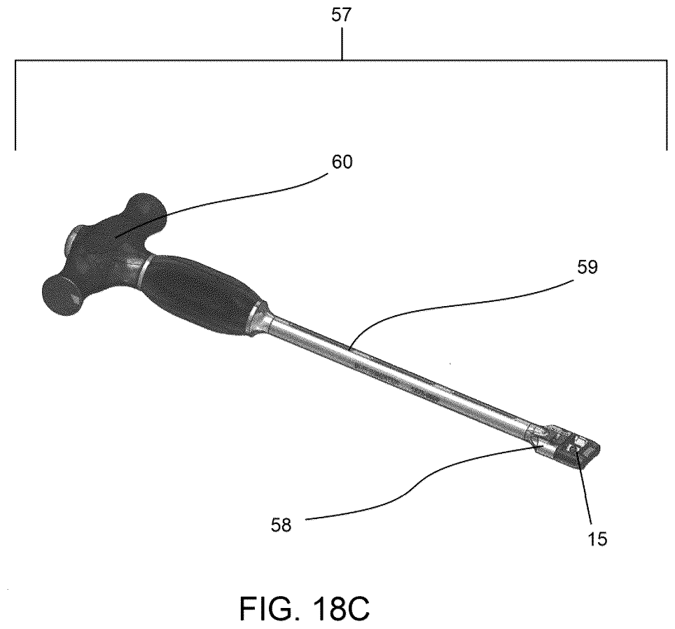

At this point, the surgical site is now fully prepared, and is now ready to accept the implant. In the preferred embodiment, the unique, useful, novel and nonobvious SIFS implant 15, which has already been described in Images 5A and 5B, is comprised of a rotating blade component 19 positioned within a frame 56. FIG. 18A is the non-deployed implant 15 reversibly coupled to the leading end 58 of the implant insertion device (the device 57 is illustrated in its entirety in FIG. 18C); the rotating blades 19 are retracted within the housing frame 56. It can be appreciated that the insertion device is provided with an external shaft 59 within which the actuating, rotatable shaft is found, the leading end 62 of which can be seen exiting from the external shaft 59 and reversibly coupling with the implant 15; the coupling itself is obscured owing to this projection. The junction of the implant 15 and the leading end 58 of the insertion device is demarcated by a line 63.

Deployment of the SIFS 15, as shown in FIG. 18B, involves rotating the blade component 19, in which one blade component 20 is rotated into the sacrum and the other 20a is rotated into the ilium. This is accomplished by insertion of the leading end of the actuating shaft of the 35 insertion device (not shown here) into the insertion area 25 at the trailing end 24 of the central axis 23 of the implant 15. This axis 23 (the position of which is indicated by the black interrupted line) is positioned within a central channel 64 at the midportion of the rotatable blade component 19, as shown by the interrupted white lines, and upon actuation of the trailing end 24 of the central axis 23, the interface between the axis 23 and the rotating component 19 will compel the rotating component into the final deployed position. It is recognized that the rotation of the blades also harvests the bone graft for fusion substrate, and when the blades are brought into final position, this monolithic piece which spans the joint space serves as a stabilization device.

The insertion device 57 is portrayed in FIG. 18C, whereupon it is noted that this is comprised of a leading end 58, which interfaces with the SIFS 15 and in fact is demonstrated here reversibly coupled to an implant that has been positioned for implantation and deployment. There is also a central shaft 59 which provides the needed length to the instrument and communicates the leading end with the trailing end 60, which has been provided with an actuating handle which provides the rotational input to deploy the implant 15. The relationship of 36 the inner actuating shaft which is the shaft that is actually actuated by the movement of the handle has already been reviewed above.

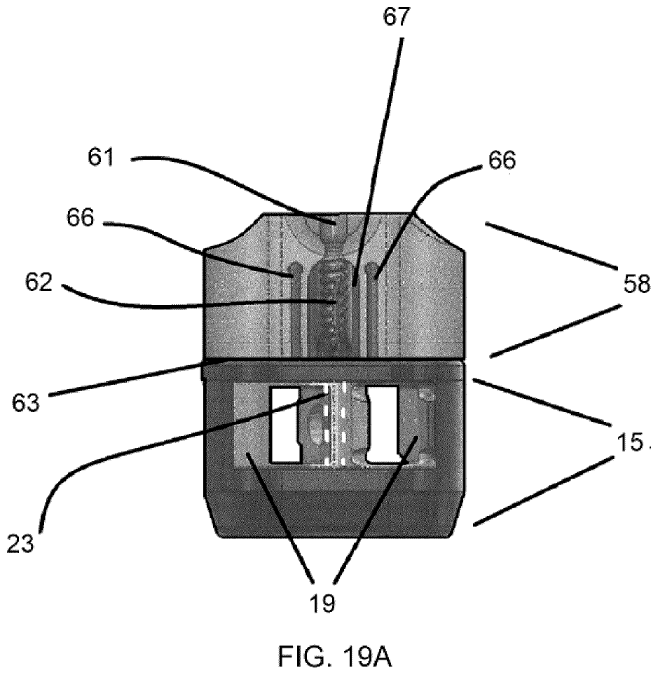

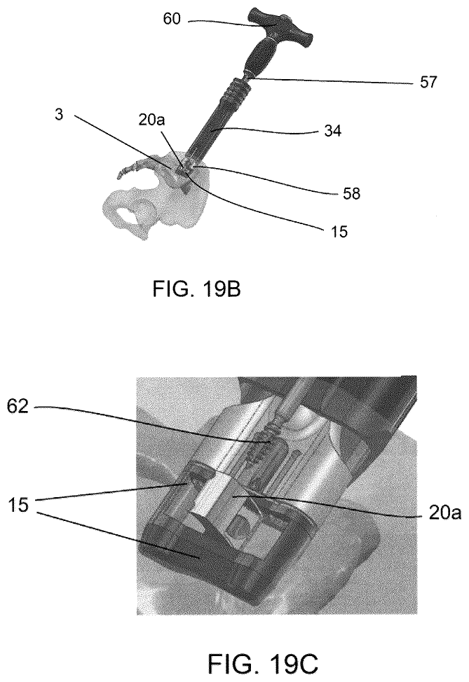

This mechanism by which the leading end 58 of the insertion instrument can be more fully appreciated in FIG. 19A, a lateral perspective of the implant being deployed within the joint space. In this image, the central portion of the leading end 58 has been cut away to demonstrate the complete mechanism by which actuation of the insertion device results in deployment of the implant 15. One notes that the leading end 62 of the rotatable shaft 61 is threaded so as to create additional torque. This is encased within a chamber 67 that provides resistance to the threading, further adding to the torque. Securing bolts 66 further strengthens the leading end 58. The leading end 62 of the actuating shaft 61 then interfaces with the central axis 23 (again indicated by the interrupted white line), which then compels the rotatable component 19 to deploy.

This is shown as it would be reduced to practice in FIG. 19B, in which a lateral view of the pelvis with the right Ilium subtracted shows the insertion instrument 57 having been disposed through the working cannula 34 so as to insert the implant 15 into the right SIJ 3

The implant 15 is seen being deployed within the target SIJ in FIG. 19C. The leading end 62 of the actuating rod has rotated the central axis of the deployable component, and the iliac blade 20a is seen directed out of the page towards the reader. This would be within the substance of the ilium if the image of the ilium had not been subtracted for this Figure.

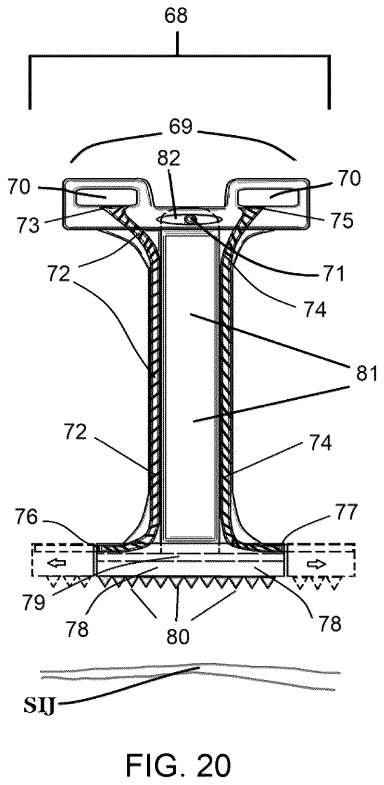

An alternative embodiment of the system of removing the cartilage from the joint as well as decorticating the bone is elucidated in FIG. 20. Here, a unique, useful, novel and nonobvious device hereinafter referred to as the Joint Debrider 68 is shown to be comprised of an actuator handle 69 at the trailing end of the instrument, a stabilizing/transmission shaft 81, and a leading end comprised of a mobile element hereinafter known as the rider 78. This mobile unit 78 is mounted on a track 79 which is continuous with and orthogonal to the transmission shaft 81, conferring upon the mobile unit 78 a range of movement along the axis described by this track 79. The movement is driven by a pair of flexible transmission elements 72, 74 which function primarily as cable-like elements. These are comprised of flexible plastic or another substance known or acceptable to the art. These elements 72, 74 extend from the trailing end of the device 68 to the leading end 78, and transmit movements from the actuator handle 69 at the trailing end to the leading end. The actuator handle 69 is provided 38 with apertures 70 designed to accommodate grasp between fingers; additionally, the handle is mounted on rotational mechanism at the trailing end of the transmission shaft 81. This mechanism consists of an axle 71 passing through an aperture 82 in the handle 69, this configuration according a rocking movement on the handle 69. With this movement, the interfaces 73, 75 between the handle 69 and the transmission elements 72, 74 (striped elements distinguish them in this image) is then transmitted along the course of the transmission elements, ultimately interfacing 76, 77 with the ends of the mobile unit 78. As the handle 69 is actuated in a rocking, “up and down,” movement, the transmission elements are, as the result of the geometry of the interface 76, 77 of the elements 72, 74 with the mobile unit 78, the unit oscillates in a direction perpendicular to the stabilization shaft. This movement would be along the axis of the target SIJ, indicated by the gray irregular line. The teeth 80 on the leading edge of the device 68 accomplish the object of opening the joint and removing cartilage and bone. It should be noted that other mechanisms which could successfully actuate this device include pneumatic/hydraulic mechanisms, magnetic drive, and power drive. None of the iterations herein described or referred to should be construed as inclusive, and any other iterations anticipated by those skilled in the art are within the spirit and scope of this application.

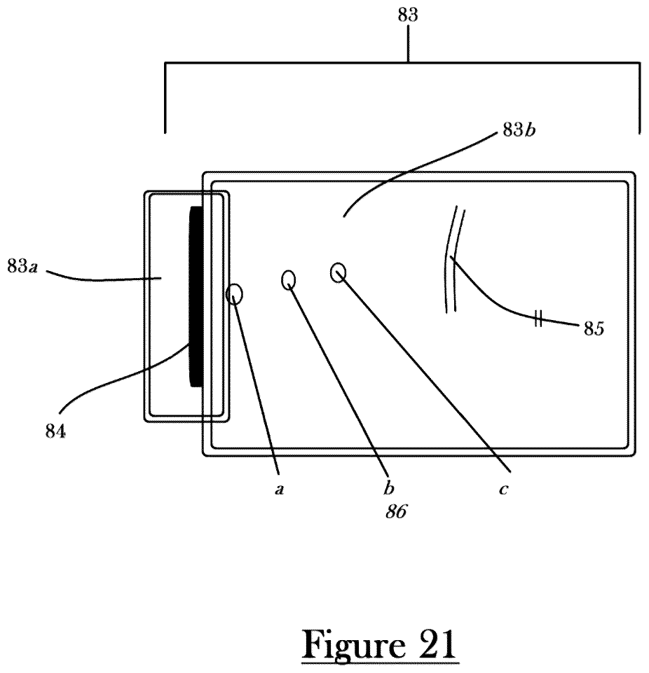

The surgical approach can be achieved in a number of ways including the “free hand,” method in concert with fluoroscopy or CT scanning as well as advanced methods such as computer assisted image guided navigation. The significant challenge in achieving the posterior approach described in this specification is the obstruction created by the overhang of the posterior iliac crest. To address this challenge, one option disclosed herein is a unique, useful, novel and nonobvious template that can be utilized to direct the trajectory of the initial guide pin, which in turn is responsible for positioning the remainder of the surgical procedure. In FIG. 21, an exemplary template is demonstrated. It must be recalled that many iterations of such a template can be anticipated by those skilled in the art, such that the iteration disclosed herein cannot be construed to be inclusive, and by definition any and all such iterations are included within the spirit and scope of this application. In this example, the template 83 is composed of two clear, sterile sheets, 83a, b presumably fabricated from plastic or other substance acceptable to the art. These sheets are slidably coupled with each other, so that landmarks which will be identified by markings which have been provided to the template 83 can be properly aligned. The template 83 is introduced into the sterile operative field and laid against the skin of the patient's back. One of the template sheets 83a is provided with a prominent black line 84 comprised of a radiopaque material. This is known as the midline market, and the surgeon utilizes 40 fluoroscopy to align this marker with the radiologic midline. The second sheet 83b is also provided with a radiopaque marker 85, in this instance it is known as the SIJ marker. The two lines 85 are ideally aligned with the target joint. The challenge is that this joint is typically best seen with the patient partially tilted with the target side down. Alternatively, an oblique radiographic view with the patient in the prone position can reveal the joint. When the two markers are aligned, then the apertures 86a, b, c will dictate the entry point, and to some degree the trajectory. It can be expected that there would be at least 3 apertures, corresponding with the basic body types (ectomorph, mesomorph, endomorph), but other configurations of these apertures can also be envisioned.

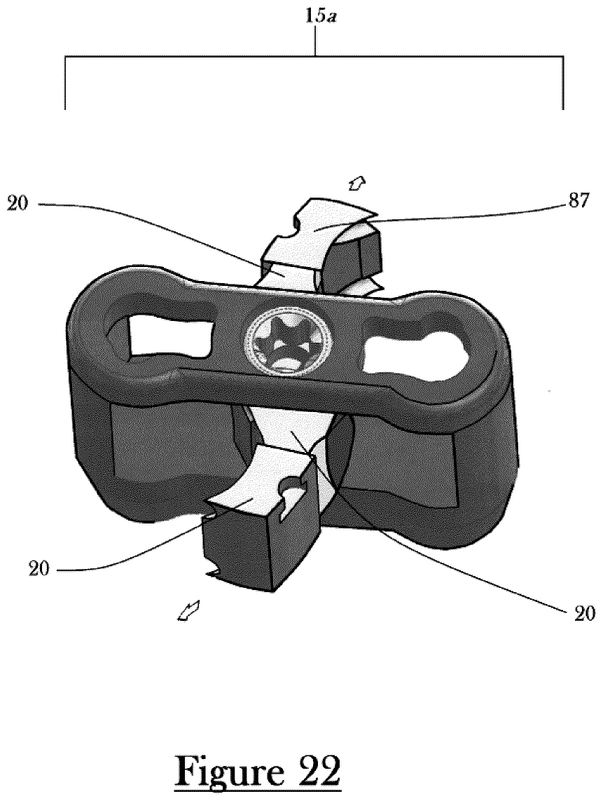

Multiple embodiments of the SIFS implant can be conceived. In one 15a, described in FIG. 22, is an embodiment of the SIFS with a cage carrier and a dual block blade with two side blades extending from a central axis engager and a connecting blade between the two side blades. The general configuration of the SIFS is retained but extensions 87 have slidably, irreversibly coupled to the blades 20 so that upon deployment a greater penetration of the cancellous bone is achieved. The open arrows indicate the direction of the repositioning of the extensions.

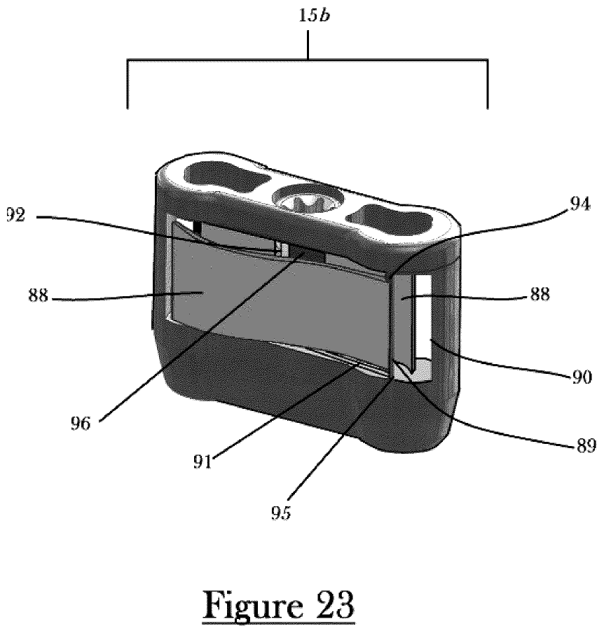

Another embodiment 15b is portrayed in FIG. 23. This embodiment also attempts to provide blades which extend a greater 41 distance into the cancellous bone of the ilium and sacrum, which will presumably reduce the amount of translational/rotational or “rocking” movements of the implant. This likely would also increase the amount of bone harvested for a fusion, and thereby increase the chances of a fusion. Given the fact that the stabilization is being achieved—to some degree—by the depth of bone penetration, then increasing the length of the blades achieves this object. In this embodiment, the blades 88 extend almost the entire length of the implant 15b by occupying the sides of the implant 15b. Each blade 88 is provided with a free end 93; the opposite end of each blade 88 terminates in an anchor 89, which is an enlargement of the blade with extensions 94 into the superior aspect of the implant 15b as well as extensions 95 into inferior aspect. These extensions 94, 95 terminate in tracks 91 in the implant 15b which guide the blades 88 when they are deployed. This occurs by slidably repositioning the free ends 93 of the blades 88 through the reduced ends 90 of the implant 15b until the anchors 89 couple with an anchoring frame 92 which is monolithic with the central axis 96 of the implant. With this coupling, the free ends 93 are partially protruding through the ends of the implant. In one embodiment, not illustrated here, the free ends of the blades 88 are curved into somewhat of a “scoop,” configuration, so as to obtain more bone graft upon deployment. Regardless of the configuration of the blades, upon the coupling of the 42 anchors with the anchor frame 92, and the central axis 96 is rotated 90° thus driving the free ends 93 of the blades 88 into their respective bony targets.

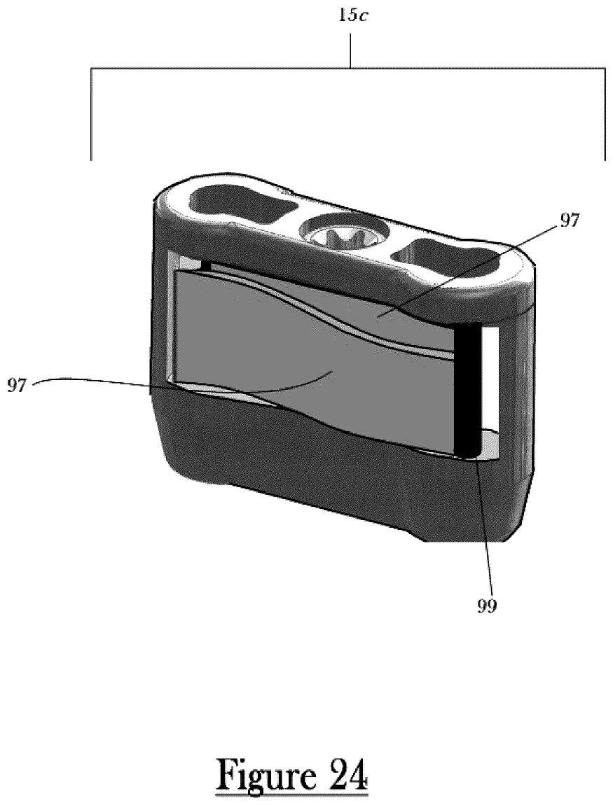

In FIG. 24, another variation of the blades 97 results in another embodiment 15c. Here, the leading ends 98 blades 97 are curved outwards (opposite the direction of the curvature in FIG. 23) and are anchored 99 at the ends of the implant 15c. There is no need for a central axis, as in other embodiments; however, at the trailing end of the implant, a complex gear mechanism (not shown here) which will rotate the anchors of the blades, hence rotating these [longer] blades into the cancellous bone.

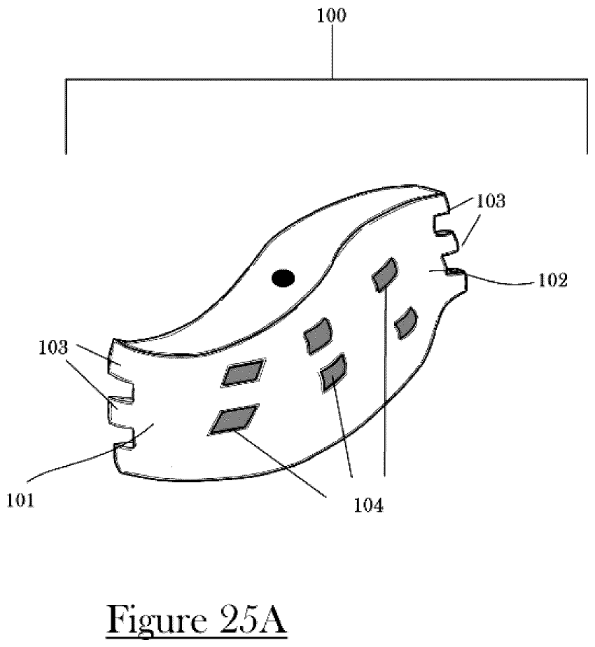

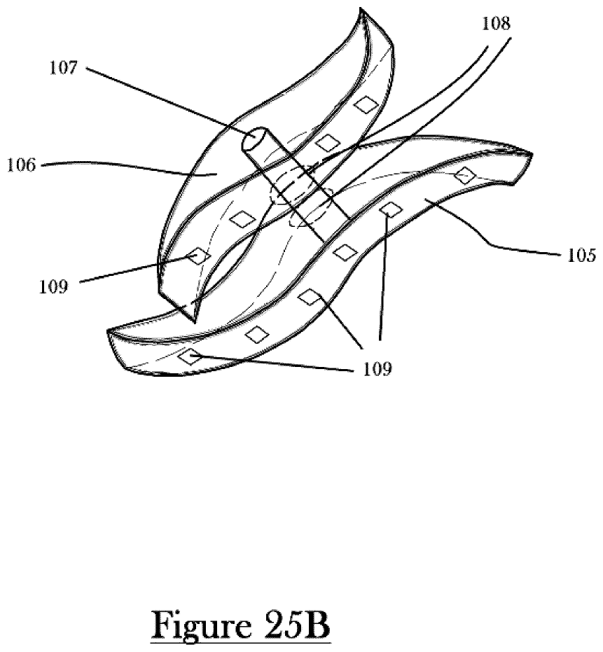

Embodiments of the SIFS which do not include a frame would involve introducing an embodiment similar to the rotatable component of the preferred embodiment of the SIFIS after subtracting the frame of the SIFS. Such embodiments are illustrated in FIGS. 25A/B.

In FIG. 25A, a version of the SIFS 100 which would be “free standing,” inasmuch that no external frame would be utilized demonstrates that this would have a substantially “sigmoid,” configuration as viewed from the top. This design 43 would favor the implantation such that after preparation of the joint, the long axis of the SIFS 100 is aligned with the long axis of the target SIJ. Then the SIFS 100 is rotated approximately 90°, with the gentle curves of the ends 101, 102 of the implant 100, favoring the passage into the cancellous bone; additionally, the curvatures of the ends 101, 102 will promote osseous material into the prepared area of the joint, as the geometry will “scoop,” this osseous material in that direction. The ends 101, 102 are also provided with projections 103 which will promote stabilization of the implant 100 into its final position. The sides of the implant are provided with apertures 104, which lead into a central chamber and are ultimately continuous with apertures on the other side. Such a design would promote growth through the embodiment itself, thus further promoting the fusion

A version can utilize 2 sigmoid-shaped blades illustrated in FIG. 25B. These would have deep 105 and superficial 106 stabilizers, aligned with each other at the time of implantation. A central axis 107 serves to irreversibly couple these two stabilizers with each other by passing through the central portion of both of stabilizers. A plurality of coupling units 108 irreversibly couples the axis 107 to the stabilizers 105, 106 in a manner that this coupling does not retard the rotational movements of the stabilizers. Again, the curvature of the ends of 44 the stabilizers will presumably promote their passage into the target areas of cancellous bone. The projections which are found at the ends of the embodiment portrayed in FIG. 25A can also be added in this embodiment, although not illustrated here. Apertures 109 on the sides of the stabilizers again will presumably promote bony growth through these units. It is anticipated that in this embodiment, it would initially be introduced with both stabilizers aligned with each other and along the long axis of the joint; it is anticipated that the width of the joint has been slightly increased by the joint preparation measures previously disclosed. After passing this embodiment into the joint space, the stabilizers are rotated in opposite directions. With the orientation of the curved ends of the stabilizers 105, 106 rotating in opposite directions ensures maximum impaction into the bone by assuming an “X” configuration providing multiple points of bone anchoring. Not pictured here is a C-clamp type device, or any other similar device, can be disposed over the top of the deployed embodiment as a locking technique. In the embodiment portrayed herein, the deep stabilizer 105 is slightly larger than the more superficial stabilizer 106, based on the concept that placing a larger construct deeper may reduce backout; obviously, this arrangement could be reversed, or the stabilizers could be of equal size, with all such embodiments incorporated within the spirit and scope of the invention.

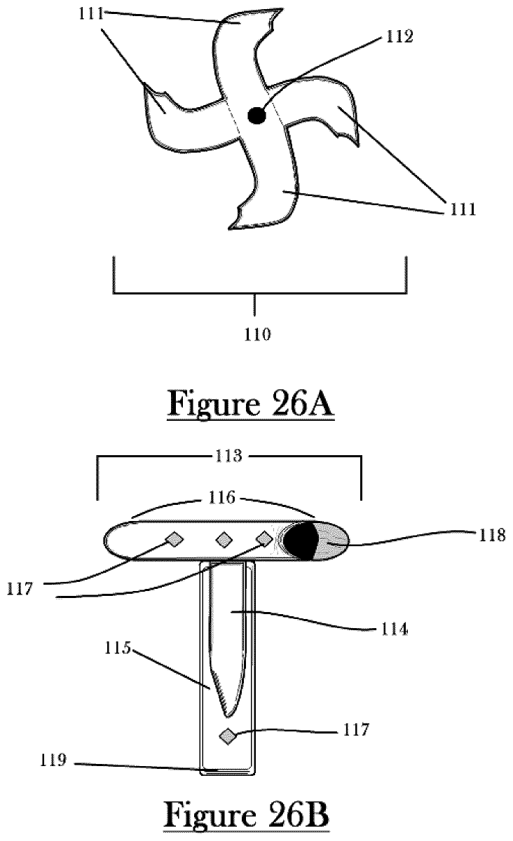

Additional variations are illustrated in FIGS. 26A and 26B. In the first of these, a top view of a SIFS 110 with 4 arms 111 is seen. This entire unit may be monolithic and impacted into the joint space after preparing the joint space to receive the unit in a “Cross” configuration and then rotating it into the final “X” configuration portrayed herein. The sides of the arms are not seen in this projection, but apertures may again be provided to encourage bony ingrowth and fusion. Alternatively, as suggested by the interrupted lines and central axis 112, the two pairs of arms might initially be aligned and upon impaction into the joint, one set is then rotated into final position.

Another embodiment which could address some of the goals of an ideal SIFS would also be provided with 4 arms. However, in this embodiment 113, seen in a lateral perspective, two of these arms are retracted against the body 115 in the primary position. One such arm 114 is seen in this view, while a similar arm on the other side of the body 115 is obscured from view in this projection. Orthogonal to the trailing end of the body 115 are the fixed arms 116, the leading end of one being seen end-on with a large aperture 118 which leads to the chamber within the center of this element. Apertures 117 on the side of the arms and body will further promote bony ingrowth. It is anticipated that this embodiment of the SIFS 113 is impacted into the prepared joint space with the leading end 119 first, 46 and the deployable arms in the primary position, and the fixed arms 116 being implanted parallel to the long axis of the joint. The deployable arms are then brought into the functional position, in this view maximally extended into the plane of the reader's view, and orthogonal to the fixed arms 116 as well as the body 115. The SIFS 113 is then rotated approximately 30-45°, completing the stabilization. As in all of the embodiments disclosed in this specification, this SIFS 113 can be filled with bone graft substrate before or after implantation.

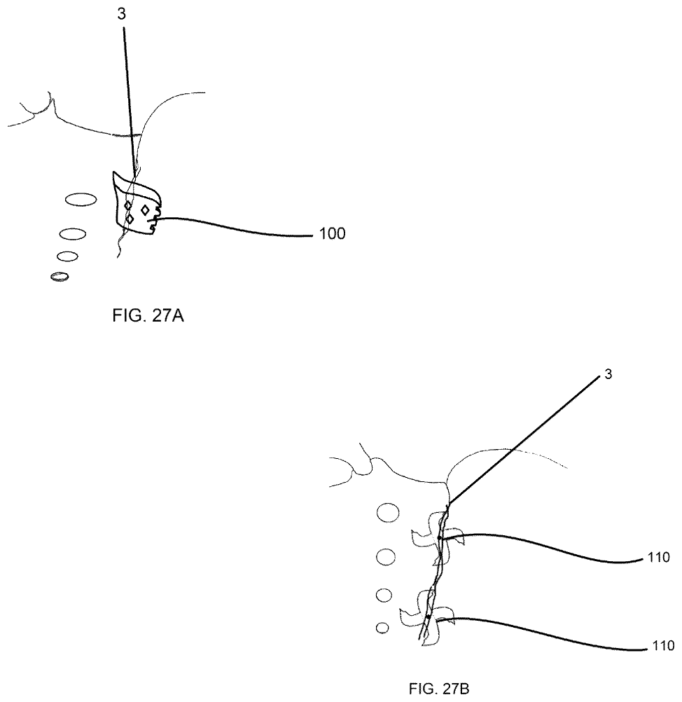

FIG. 27A is a posterior schematic view of the right side of the sacrum, the right SIJ 3 and the medial aspect of the ilium. This shows the final position of the frameless embodiment 100 of the SIFS locked into its final position. In FIG. 27B it is demonstrated that two SIFS units can be utilized in the fusion and stabilization. In this illustration, an embodiment 110 with 4 arms is used; however, this is exemplary, and any of the SIFS embodiments disclosed in this specification could be used in the same way.

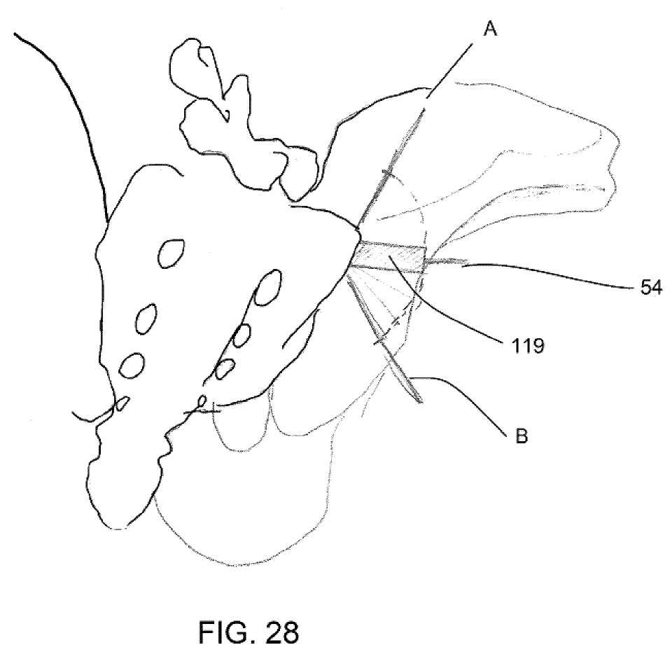

In the setting of multiple implants, the implantation method described above could result in successful placement of the SIFS devices. Alternatively, as shown in FIG. 28, a different method of 47 implantation can be utilized. The working cannula 119 has been modified such that there is a movable joint on the leading end, therefore allowing the drill 54 to adjust the trajectory cranially and caudally, thus preparing these two areas for implantation. Then SIFS implants are disposed through the working cannula and implantation is completed.

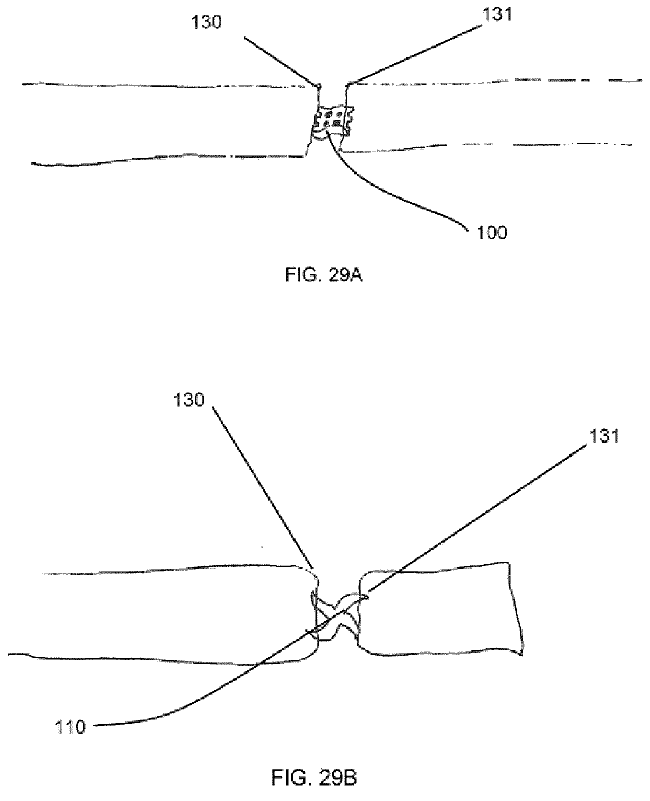

The SIFS can be used to treat non-healing fractures/non-union, particularly of the long bones. Drilling out the area of pseudoarthrosis and implanting the SIFS would likely provide a significantly high chance of healing of the bony fractures. This is illustrated in FIGS. 29A/B. In both of these illustrations, the fractured ends of the long bones 130, 131 have not healed leaving an area of pseudoarthrosis. In FIG. 29A, the embodiment of the SIFS 100 which has no frame and is provided with 2 arms is utilized. In FIG. 29B, the 4 arm embodiment 110 is utilized. These illustrations do not exclude the possibility of using an embodiment with a frame.

What is claimed is:

1. An apparatus for fusing the sacroiliac joint comprising:
A rectangular cage with a top segment, a bottom segment, a first major side, a second major side, a first minor end and a second minor end, where said rectangular cage has a coating to promote bone adhesion and said top segment has a first axis bearing opening and said bottom segment has a second axis bearing opening;
An axle having a major axis body with a top portion and bottom portion held by said first axis bearing opening and said second axis bearing opening;
At least two blades disposed on opposite sides of said axle projecting orthogonally from said axle where said blades are sinusoidal when viewed from the top of said axle.

2. The apparatus of claim 1 where said blade has a top blade and a bottom blade extending from said axle, and where said top blade and said bottom blade.

3. The apparatus of claim 2 wherein said top blade and said bottom blade are joined at their outermost point by an orthogonal connecting blade.

4. An apparatus for fusing across the sacroiliac joint comprising:
a leading end 16, a central deployable segment 17, and a trailing end 18; the leading end 16 is tapered so and plasma coated;
a rotating component 19 rotates around a central axis with each end further provided with blades 20 designed to cut into the cancellous bones of the ilium and sacrum; said blades have apertures 21 positioned within these blades 20 direct the cancellous bone;
the central axis has an actuator end that actuates the rotating component 19 of the implant 15 wherein actuation the blades 20 27 of the rotating component 19.

5. An apparatus for fusing at least two bone segments comprising:
A central body with a first aperture and a second aperture window joined by an inner chamber;
A first arm and a second arm retracted against into said cavity of said central body 115 in the primary position and orthogonal to the trailing end of the body 115 are two fixed arms.

6. The apparatus of claim 5 wherein said apertures are provided to 117 the side of the arms and body.
